(19) United States
(12) Patent Application Publication
(10) Pub. No.: US 2025/0111891 A1
(43) Pub. Date: Apr. 3, 2025

(54) METHOD, APPARATUS, ELECTRONIC DEVICE AND STORAGE MEDIUM FOR DEVICE FOR GENERATING LIGAND MOLECULE

(71) Applicants: Douyin Vision Co., Ltd., Beijing (CN); Lemon Inc., Grand Cayman (KY)

(72) Inventors: Yuwei YANG, Los Angeles, CA (US); Xiangxin ZHOU, Beijing (CN); Xiwei CHENG, Beijing (CN); Yu BAO, Beijing (CN)

(21) Appl. No.: 18/897,735

(22) Filed: Sep. 26, 2024

(30) Foreign Application Priority Data
Sep. 28, 2023 (CN) 202311277957.6

(51) Int. Cl. G16B 15/30; G06F 30/27; G16B 15/20; G16B 40/20

(52) U.S. Cl. CPC G16B 15/30; G06F 30/27; G16B 15/20; G16B 40/20

(57) ABSTRACT

The embodiment of the invention provides a method, apparatus, device and storage medium for generating ligand molecules. The method comprises: determining a set of initial arms by decomposing a reference ligand molecule for a target biological target; generating a set of candidate ligand molecules based on the set of initial arms; determining a set of candidate arms corresponding to each of the set of initial arms by decomposing each of the set of candidate ligand molecules; determining a target candidate arm for each initial arm from the set of candidate arms to determine a set of target candidate arms; and generating a set of ligand molecules for the target biological target based on the set of target candidate arms. In this way, by selecting the key information (i.e., the arm) more fitting the drug design target in the drug design process as a condition, the embodiments of the present disclosure can improve the ratio of the candidate drug molecules to the drug design target.

### CROSS-REFERENCE TO RELATED APPLICATIONS

This application claims priority to Chinese Patent Application No. CN202311277957.6, filed on Sep. 28, 2023, and entitled “METHOD, APPARATUS, ELECTRONIC DEVICE AND STORAGE MEDIUM FOR DEVICE FOR GENERATING LIGAND MOLECULE”, the entirety of which is incorporated here by reference.

### FIELD

Example embodiments of the present disclosure generally relate to the field of computers, and in particular, to method, apparatus, electronic device, and computer-readable storage media for generating ligand molecule.

### BACKGROUND

The interaction between biomolecules is an important basis for its biological activity. For example, a human body may produce an antibody protein in combination with an invasive virus to inhibit the disease. In biopharmaceutical research, physical and chemical mechanisms of inter molecular interactions can be understood by analyzing those molecules known to be bound to each other, thereby helping to design novel drug molecules that can be combined with some specific targets. For example, the ligand molecule in combination with the target protein may be determined, and then the drug design is performed based on the ligand molecule. Therefore, how to efficiently determine a ligand molecule capable of binding to a target protein is one of the problems that need to be solved at present.

### SUMMARY

In a first aspect of the present disclosure, a method for generating a ligand molecule is provided. The method includes: determining a set of initial arms by decomposing a reference ligand molecule for a target biological target; generating a set of candidate ligand molecules based on the set of initial arms; determining a set of candidate arms corresponding to each of the set of initial arms by decomposing each of the set of candidate ligand molecules; determining a target candidate arm for each initial arm from the set of candidate arms to determine a set of target candidate arms; and generating a set of ligand molecules for the target biological target based on the set of target candidate arms.

In a second aspect of the present disclosure, a method for constructing a ligand generative model is provided. The method comprises obtaining a biological target ligand molecule pair, the biological target ligand molecule pair comprising a training biological target and a corresponding training ligand molecule; determining a set of training arms by decomposing the training ligand molecules; updating an atomic feature representation of a plurality of atoms corresponding to the set of training arms in the training ligand molecule based on a set of arm feature representations of the set of training arms; determining a target feature representation of the biological target ligand molecule pair based on the updated atomic feature representation; and training a ligand generative model using the target feature representation of the biological target ligand molecule pair.

In a third aspect of the present disclosure, an apparatus for generating a ligand molecule is provided. The apparatus comprises a first decomposition module configured for determining a set of initial arms by decomposing a reference ligand molecule for a target biological target; a first generation module configured for generating a set of candidate ligand molecules based on the set of initial arms; a second decomposition module configured for determining a set of candidate arms corresponding to each of the set of initial arms by decomposing each of the set of candidate ligand molecules; a first determination module configured for determining a target candidate arm for each initial arm from the set of candidate arms to determine a set of target candidate arms; and a second generation module configured for generating a set of ligand molecules for the target biological target based on the set of target candidate arms.

In a fourth aspect of the present disclosure, an apparatus for constructing a ligand generative model is provided. The apparatus comprises a first obtaining module configured for obtaining a biological target ligand molecule pair, the biological target ligand molecule pair comprising a training biological target and a corresponding training ligand molecule; a third decomposition module configured for determining a set of training arms by decomposing the training ligand molecules; a feature updating module configured for updating an atomic feature representation of a plurality of atoms corresponding to the set of training arms in the training ligand molecule based on a set of arm feature representations of the set of training arms; a second determination module configured for determining a target feature representation of the biological target ligand molecule pair based on the updated atomic feature representation; and a model training module configured for training a ligand generative model using the target feature representation of the biological target ligand molecule pair.

In a fifth aspect of the present disclosure, an electronic device is provided. The device includes at least one processing unit; and at least one memory coupled to the at least one processing unit and storing instructions for execution by the at least one processing unit. The instructions, when executed by the at least one processing unit, cause the electronic device to perform the method of the first aspect or the second aspect.

In a sixth aspect of the present disclosure, a computer-readable storage medium is provided. The medium stores a computer program, and when the computer program is executed by the processor, the method in the first aspect or the second aspect is implemented.

It should be understood that the content described in this section is not intended to limit the key features or important features of the embodiments of the present disclosure, nor is it intended to limit the scope of the present disclosure. Other features of the present disclosure will become readily understood from the following description.

### BRIEF DESCRIPTION OF DRAWINGS

The above and other features, advantages, and aspects of various embodiments of the present disclosure will become more apparent from the following detailed description taken in conjunction with the accompanying drawings. In the drawings, the same or similar reference numbers refer to the same or similar elements, wherein:

FIG. 1 illustrates a schematic diagram of an example environment in which embodiments of the present disclosure can be implemented;

FIG. 2 illustrates a flowchart of an example process of constructing a ligand generative model according to some embodiments of the present disclosure;

FIG. 3 illustrates a schematic diagram of molecular decomposition according to some embodiments of the present disclosure;

FIG. 4 illustrates a flowchart of an example process of generating a ligand molecule according to some embodiments of the present disclosure;

FIG. 5 illustrates a schematic structural block diagram of an apparatus for generating a ligand molecule according to some embodiments of the present disclosure;

FIG. 6 illustrates a schematic structural block diagram of an apparatus for constructing a ligand generative model according to some embodiments of the present disclosure; and

FIG. 7 illustrates a block diagram of an electronic device capable of implementing various embodiments of the present disclosure.

### DETAILED DESCRIPTION

Embodiments of the present disclosure will be described in more detail below with reference to the accompanying drawings. While certain embodiments of the present disclosure are shown in the accompanying drawings, it should be understood that the present disclosure may be implemented in various forms, and should not be construed as limited to the embodiments set forth herein, but rather, these embodiments are provided for a more thorough and complete understanding of the present disclosure. It should be understood that the drawings and embodiments of the present disclosure are for exemplary purposes only and are not intended to limit the scope of the present disclosure.

It should be noted that the headline of any section/subsection provided herein is not limiting. Various embodiments are described throughout and any type of embodiments may be included in any section/subsection. Furthermore, the embodiments described in any section/subsection may be combined in any manner with the same section/subsection and/or any other embodiment described in different sections/subsections.

In the description of the embodiments of the present disclosure, the terms “including” and the like should be understood to include “including but not limited to”. The term “based on” should be understood as “based at least in part on”. The terms “one embodiment” or “the embodiment” should be understood as “at least one embodiment”. The term “some embodiments” should be understood as “at least some embodiments”. Other explicit and implicit definition may also be included below. The terms “first,” “second,” and the like may refer to different or identical object. Other explicit and implicit definition may also be included below.

Embodiments of the present disclosure may relate to data of a user, acquisition and/or use of data, and the like. These aspects all follow the corresponding laws and regulations and related regulations. In the embodiments of the present disclosure, all data is collected, obtained, processed, processed, forwarded, used, etc., all of which are performed on the premise that the user knows and confirms. Accordingly, when implementing the embodiments of the present disclosure, the types of the data or information that may be involved, the usage scope, the usage scenario, and the like should be notified to the user and obtain the authorization of the user in an appropriate manner according to the relevant laws and regulations. The specific notification and/or authorization manner may vary according to actual situations and application scenarios, and the scope of the present disclosure is not limited in this respect.

According to the solutions in the present specification and the embodiments, for example, personal information processing is involved, processing may be performed on the premise of having a legality basis (for example, obtaining consent of a personal information subject, or necessary for performing a fulfillment contract), and processing only within a specified or agreed range. The user rejects personal personal information other than necessary information required by the basic function, and does not affect the basic function of the user.

As briefly mentioned above, the ligand molecule to which it is designed for a given target spot is the important ring in the drug design. Specifically, the task aims to generate ligand molecules with certain affinity with the drug target, and the generated ligand molecules should also meet the drug properties and the synthesizability, and candidate molecules are provided for screening and optimization of subsequent drugs.

However, conventional generative model-based methods generally ignore the requirements for various properties in the actual pharmaceutical process, resulting in lower efficiency in finding potential effective drug molecules.

Embodiments of the present disclosure provide a solution for generating ligand molecules. According to the scheme, a set of initial arms can be determined by decomposing reference ligand molecules for a target biological target; a set of candidate ligand molecules are generated based on a set of initial arms; a set of candidate arms corresponding to each initial arm in a set of initial arms is determined by decomposing each candidate ligand molecule in a set of candidate ligand molecules; a target candidate arm for each initial arm is determined from a set of candidate arms to determine a set of target candidate arms; and a set of ligand molecules for the target biological target are generated based on a set of target candidate arms.

In this way, by selecting the key information (i.e., the arm) more fitting the drug design target in the drug design process as a condition, the embodiments of the present disclosure can improve the ratio of the candidate drug molecules to the drug design target.

Various example implementations of this scheme are described in detail below in conjunction with the accompanying drawings.

### Example Environment

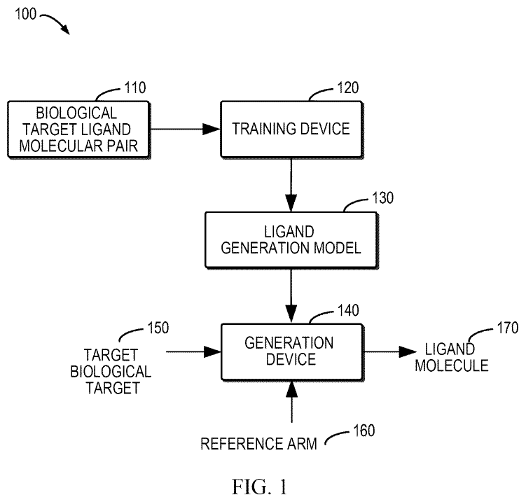

FIG. 1 illustrates a schematic diagram of an example environment 100 in which embodiments of the present disclosure can be implemented. As shown in FIG. 1, the example environment 100 may include a training device 120 and a generation device 140.

In this example environment 100, the training device 120 may obtain a target ligand molecule pair 110, which may include a training target and a corresponding training ligand molecule. The plurality of target ligand molecule pairs may construct a training set for training the ligand generation model 130.

As will be described in detail below, the training device 120 may train the ligand generation model 130 with the biological target ligand molecule pair 110 aggregated by the training set. Such a ligand generation model 130 may be a diffusion model. Exemplarily, the ligand generation model 130 may be generated based on decomposition of atoms in the ligand, which may be implemented by, for example, a decomposable diffusion (Decoffuse) model.

Further, the generation device 140 may utilize the ligand generation model 130 to complete the generation of the ligand molecules 170. Specifically, the generating device 140 may obtain the target biological target 150, and generate the ligand molecule 170 matched with the target biological target 150 by using a set of reference arms 160.

The specific generation process of the ligand molecule 170 will be described in detail below.

It should be understood that although the training device 120 and the generation device 140 in FIG. 1 are shown as two separate blocks, they may be implemented by the same or different electronic devices.

### Example Training Process

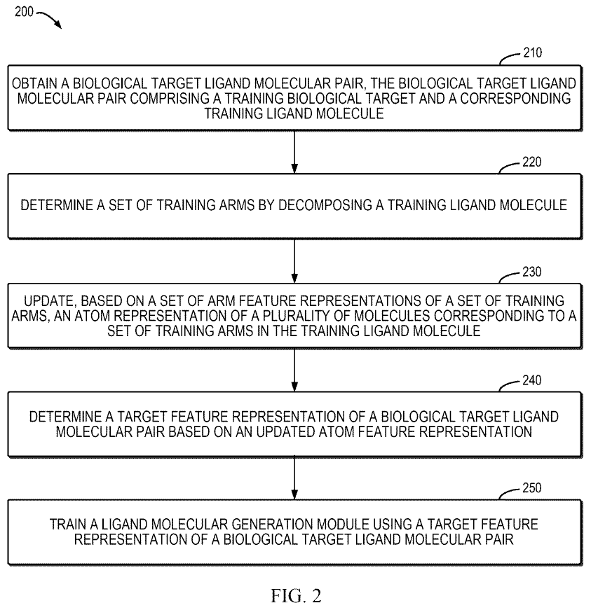

FIG. 2 illustrates a flowchart of an example process 200 of constructing a ligand generative model according to some embodiments of the present disclosure. Process 200 may be implemented at training device 120. The process 200 is described below with reference to FIG. 1.

At block 210, the training device 120 obtains a target ligand molecule pair 110, wherein the biological target ligand molecule pair includes a training target and a corresponding training ligand molecule.

Specifically, in the training phase, the training device 120 may obtain a biological target ligand molecule pair, the biological target ligand molecule pair comprising a training biological target and a corresponding training ligand molecule.

At block 220, the training device 120 determines a set of training arms by decomposing the training ligand molecules.

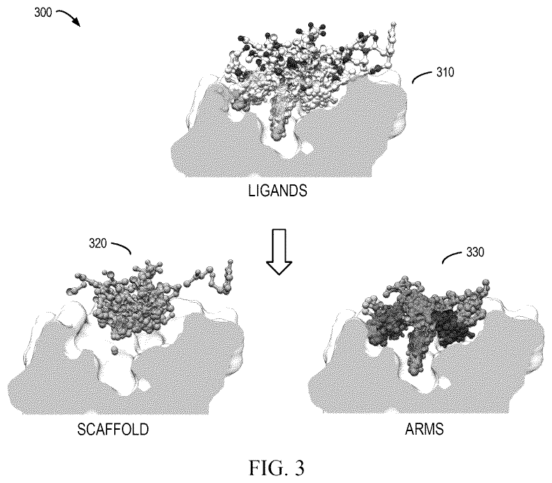

FIG. 3 shows a schematic diagram 300 of decomposed ligand molecules according to some embodiments of the present disclosure. As shown in FIG. 3, the training device 120 may decompose the ligand molecules 310 into a scaffold 320 and a set of arms 330.

Specifically, the training device 120 may divide a plurality of atoms in the training ligand molecule into a plurality of atomic clusters based on a binding between each atom in the training ligand molecule and a local binding pocket of the training target, the plurality of atomic clusters including a set of arm atom clusters and a skeleton atom cluster. Further, the training device 120 may determine a set of training arms based on a set of arm clusters.

In some embodiments, the training device 120 may determine the location of the local bonding pocket on the training target by using an existing target binding site prediction method. Exemplarily, the local bonding pocket on the training target is determined based on geometric information (such as curvature, etc.), chemical information (e.g., affinity, etc.), and the like. For example, target binding site prediction software such as Alpha Space may be used to extract the location of the local bonding pocket on the training target.

In some embodiments, the training device 120 may divide the atoms in the training ligand molecule 310 through a clustering algorithm according to the binding between each atom in the training ligand molecule and the local binding pocket on the target sample. For example, the atoms occupying the same local bond pocket may be divided into the same arm cluster (e.g., corresponding to arm 330), and the atoms that do not occupy the local bond pocket are divided into skeleton atomic clusters (e.g., corresponding to skeleton 320). For example, the plurality of divided atomic clusters may include a skeleton atomic cluster and a plurality of arm atomic clusters.

With continued reference to FIG. 2, at block 230, the training device 120 updates an atomic feature representation of a plurality of atoms corresponding to the set of training arms in the training ligand molecule based on a set of arm feature representations of the set of training arms.

Specifically, the training device 120 may construct a first atomic neighbor map based on a distance from a plurality of atoms in the training target to atoms in the first training arm, where an atomic connection in the first atomic neighbor map indicates that a distance between the two atoms is less than a first preset distance.

For example, for each atom in the first training arm, the training device 120 may construct a k-nearest heterogeneous geometric graph together with a protein sub-pocket within its surrounding 10 Å range. The nodes in the geometric graph may correspond to atoms in the first training arm or atoms in the training target that have a distance less than 10 Å from atoms in the first training arm. The edge connection in this geometry indicates that the distance between the two atoms is less than 10 Å.

In some embodiments, the training device 120 determines the first graph feature representation of the first atomic neighbor graph as the first arm feature representation of the first training arm.

Specifically, the training device 120 may, for example, use an appropriate feature extraction module to determine the first graph feature representation of the first atomic neighbor graph as the first arm feature representation of the first training arm. Exemplarily, the training device 120 may perform feature extraction on the k-nearest neighbor heterogeneous geometric map by using an equivariant neural network, to obtain the feature of the arm level.

Further, the training device 120 may update the feature of the corresponding atom in the ligand molecule by using the feature of the arm level. Specifically, for the second training arm in the set of training arms, the training device 120 may determine a set of atoms in the training ligand molecule corresponding to the second training arm. Further, the training device 120 may concatenate the second arm feature representation (i.e., the feature representation of the arm level discussed above) of the second training arm to the atomic feature representation of the set of atoms as an atomic feature representation of the set of atomic updates.

In some embodiments, for atoms in the skeleton in the training ligand molecule, the training device 120 may not perform additional special processing.

At block 240, the training device 120 determines a target feature representation of the biological target ligand molecule pair based on the updated atomic feature representation.

Specifically, the training device 120 may construct a second atomic neighbor map based on a distance between a plurality of atoms in the biological target ligand molecular pair, where an atomic connection in the second atomic neighbor map indicates that a distance between the two atoms is less than a second preset distance.

Similar to the build process of the first atomic neighbor graph, the training device 120 may construct a k-neighbor heterogeneous geometric graph along with the training target points. In this geometry, each node may correspond to an atom in a training ligand molecule or a training target. If an atom corresponds to the set of training arms, its feature representation may be determined by the initial feature representation of the spliced atom being represented by the arm level feature of the corresponding arm. Rather, the feature representation of other atoms may then be determined in any suitable manner, which is not intended to be limiting.

Further, the training device 120 may determine the second graph feature representation of the second atomic neighbor graph as the target feature representation of the biological target ligand molecule pair.

At block 250, the training device 120 trains a ligand generative model using the target feature representation of the biological target ligand molecule pair.

Specifically, the training device 120 may perform message propagation feature learning by using the target feature representation of the biological target ligand molecule. In some embodiments, the training objective is to adjust the type and coordinate of the currently generated ligand molecule according to the input training target and each arm condition according to the input training target and each arm condition, so as to reconstruct the ligand molecule in the training sample as much as possible.

In this way, embodiments of the present disclosure can extract key information (i. e. Arms) in a ligand molecule as a condition in the process of training a given target spot information generating ligand molecule, so as to support ligand molecule generation based on key information.

### Example Generation Process

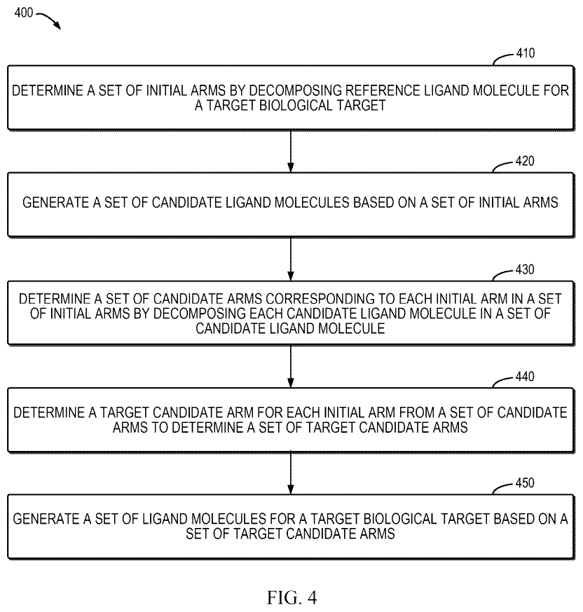

FIG. 4 shows a flowchart of an example process 400 of generating a ligand molecule according to some embodiments of the present disclosure. Process 400 may be implemented at generating device 140. The process 400 is described below with reference to FIG. 1.

As shown in FIG. 4, at block 410, the generating device 140 determines a set of initial arms by decomposing a reference ligand molecule for a target biological target.

In some embodiments, for a given target biological target, the generating device 140 may obtain a corresponding reference ligand molecule. Such reference ligand molecules may include, for example, preset ligand molecules, e.g., known ligand molecules, for the target biological target. Alternatively, such reference ligand molecules may also include reference ligand molecules generated based on other ligand molecules and generated based on the target biological target.

Further, the generating device 140 may decompose the reference ligand molecule. Specifically, the generating device 140 may divide a plurality of atoms in the reference ligand molecule into a plurality of atom clusters based on a binding between each atom in the reference ligand molecule and a local binding pocket of the target biological target, the plurality of atom clusters including a set of arm clusters and a skeleton atom cluster. Further, the generating device 140 may determine a set of initial arms based on a set of arm clusters.

For a specific decomposition process of the reference ligand molecule, refer to the process described above with respect to FIG. 3, and details are not described herein again.

At block 420, the generating device 140 generates a set of candidate ligand molecules based on the set of initial arms. Specifically, the generating device 140 may provide a set of feature representations of a set of initial arms to the ligand generative model to generate a set of candidate ligand molecules. Such a ligand generation model may be, for example, a ligand generative model trained based on the training process described in FIG. 2.

At block 430, the generating device 140 determines a set of candidate arms corresponding to each of the set of initial arms by decomposing each of the set of candidate ligand molecules.

Exemplarily, the generating device 140 may generate a predetermined number (e.g., 30) of candidate ligand molecules based on a set of initial arms, and may separately decompose the candidate ligand molecules based on the ligand molecule decomposition process described above. Thus, for each local arm, a corresponding 30 candidate arm is obtained.

At block 440, the generating device 140 determines a target candidate arm for each initial arm from the set of candidate arms to determine a set of target candidate arms.

In some embodiments, the generating device 140 may determine a target candidate arm for each initial arm from a set of candidate arms based on a property of the set of candidate arms.

As an example, such properties may include affinity, i.e., the binding between the candidate arm and the target biological target. Alternatively or additionally, such properties may also include drug properties QED for the candidate arms. Alternatively or additionally, such properties may also include synthesizability of candidate arms.

Therefore, the generating device 140 may select, from a corresponding set of candidate arms, a candidate arm whose property is better (for example, an optimal property) as the target candidate arm corresponding to the local arm. In this way, the generating device 140 may determine a set of target candidate arms corresponding to a set of initial local arms, and such set of target candidate arms may have better properties.

At block 450, the generating device 140 generates a set of ligand molecules for the target biological target based on the set of target candidate arms.

Further, the generating device 140 may provide the feature representation of the set of target candidate arms with optimized properties to the ligand generative model for generating a set of ligand molecules for the target biological target.

In some embodiments, the generating device 140 may, for example, directly use one or more ligand molecules generated based on the set of target candidate arms as final ligand molecules.

Alternatively, the generating device 140 may also generate the final ligand molecule through an iterative optimization process. Specifically, the generating device 140 may iteratively perform the following steps until the preset condition is satisfied: generating a second set of candidate ligand molecules based on a set of target candidate arms; determining a second set of candidate arms corresponding to each of the set of initial arms by decomposing each of the second set of candidate ligand molecules; and determining a target candidate arm for each initial arm from the second set of candidate arms to replace a set of target candidate arms.

Exemplarily, in the case that the second set of candidate ligand molecules are generated based on the set of candidate arms, such a second set of candidate ligand molecules may be further decomposed to obtain a new set of candidate arms. Based on a similar manner, the generating device 140 may determine a candidate arm having a better property (for example, an optimal property) among the set of new candidate arms as a new generation condition.

In some embodiments, the preset condition for terminating the iteration may include the number of iterations reaching a predetermined number of times. Alternatively, the preset condition for terminating the iteration may include that the performance of the second set of candidate ligand molecules generated in a certain iteration is higher than a threshold.

Further, the generating device 140 may generate a set of ligand molecules for the target biological target based on the set of target candidate arms after iteration.

Therefore, according to the embodiment of the invention, the key information (arms) of the drug design target more matched with iteration iteration selection can be sampled in multiple rounds in the drug design process as a condition, so that the proportion of the candidate drug molecules meeting the drug design target is continuously improved.

Further, by verifying the ligand molecule generation scheme of the present disclosure on the CrossDocked2020 dataset, the obtained experiment result shows that the ligand molecule generated by the ligand molecule generation solution according to the embodiments of the present disclosure has stronger affinity with the target point and stronger drug rationality, and meets the success rate of drug design objectives.

Specifically, the affinity of the ligand molecules generated by the ligand molecule generation solution according to the embodiments of the present disclosure is stronger. Based on indexes such as Vina Docking Score, QED, SA and the like, affinity, drug resistance and synthesizability of generated ligand molecules and targets are evaluated respectively, on a CrossDocked2020 data set, the SA scores of the Vina Docking Score, the QED and the 0.73 of the average −7.49 can be obtained through the invention, and the method is superior to a traditional comparison scheme.

In addition, the ligand molecules generated by the ligand molecule generation solution according to the embodiments of the present disclosure meet the success rate of drug design objectives. By comprehensively considering the ratio of the specified affinity and the drug rationality index threshold in the candidate molecule, that is, Success rate, the Success rate of the candidate ligand molecule generated by using the ligand molecule generation solution of the embodiment of the present disclosure is far better than that of the previous drug design algorithm.

In addition, on the data set, the average affinity of ligand molecules generated by using the ligand molecule generation scheme of the embodiments of the present disclosure is higher than that of a known traditional 3D molecule generation method, and there is a significant improvement in the success rate index considering drug resistance, synthesizability and affinity at the same time.

### Example Apparatus and Apparatus

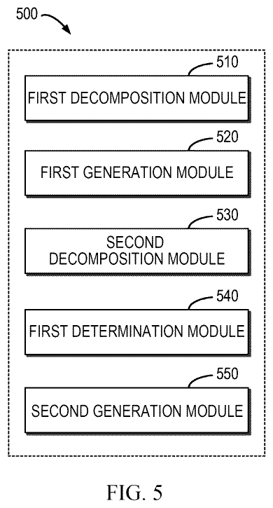

Embodiments of the present disclosure also provide a corresponding apparatus for implementing the above method or process. FIG. 5 shows a schematic structural block diagram of an apparatus 500 for generating a ligand molecule according to some embodiments of the present disclosure. The apparatus 500 may be implemented or included in the generation device 140. The various modules/components in the apparatus 500 may be implemented by hardware, software, firmware, or any combination thereof.

As shown in FIG. 5, the apparatus 500 comprises: a first decomposition module 510 configured for determining a set of initial arms by decomposing a reference ligand molecule for a target biological target; a first generation module 520 configured for generating a set of candidate ligand molecules based on the set of initial arms; a second decomposition module 530 configured for determining a set of candidate arms corresponding to each of the set of initial arms by decomposing each of the set of candidate ligand molecules; a first determination module 540 configured for determining a target candidate arm for each initial arm from the set of candidate arms to determine a set of target candidate arms; and a second generation module 550 configured for generating a set of ligand molecules for the target biological target based on the set of target candidate arms.

In some embodiments, the first generation module 520 is further configured for providing the set of feature representations of the set of initial arms to a ligand generative model to generate the set of candidate ligand molecules, wherein the ligand generation model is a trained diffusion model.

In some embodiments, the reference ligand molecule comprises: a preset ligand molecule for the target biological target; or a reference ligand molecule generated based on the target biological target according to another ligand molecules generation process.

In some embodiments, the first decomposition module 510 is further configured for: dividing a plurality of atoms in the reference ligand molecule into a plurality of atom clusters based on a binding between each atom in the reference ligand molecule and a local binding pocket of the target, the plurality of atom clusters comprising a set of arm atom clusters and a skeleton atom cluster; and determining the set of initial arms based on the set of arm clusters.

In some embodiments, the first determining module 540 is further configured for determining the target candidate arm for each initial arm from the set of candidate arms based on a property of the set of candidate arms.

In some embodiments, the property comprises at least one of: affinity, drug resistance, and synthesizability.

In some embodiments, the set of candidate ligand molecules comprise a first set of candidate ligand molecules, the set of candidate arms comprise a first set of candidate arms, and the second generation module 550 is further configured for: iteratively performing the following steps until a preset condition is satisfied: generating a second set of candidate ligand molecules based on the set of target candidate arms; determining a second set of candidate arms corresponding to each of the set of initial arms by decomposing each of the second set of candidate ligand molecules; determining a target candidate arm for each initial arm from the second set of candidate arms to replace the set of target candidate arms; generating the set of ligand molecules for the target biological target based on the set of target candidate arms after iteration.

In some embodiments, the preset condition comprises: the step is performed a predetermined number of times; or performance of the second set of candidate ligand molecules is higher than a threshold.

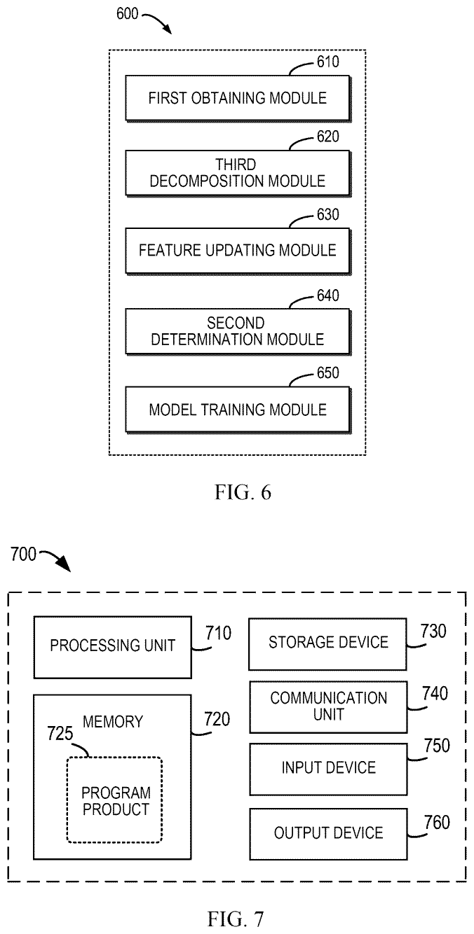

FIG. 6 shows a schematic structural block diagram of an apparatus 600 for constructing a ligand generative model according to some embodiments of the present disclosure. The apparatus 600 may be implemented or included in the training apparatus 120. The various modules/components in the apparatus 600 may be implemented by hardware, software, firmware, or any combination thereof.

As shown in FIG. 6, the apparatus 600 comprises: a first obtaining module 610 configured for obtaining a biological target ligand molecule pair, the biological target ligand molecule pair comprising a training biological target and a corresponding training ligand molecule; a third decomposition module 620 configured for determining a set of training arms by decomposing the training ligand molecules; a feature updating module 630 configured for updating an atomic feature representation of a plurality of atoms corresponding to the set of training arms in the training ligand molecule based on a set of arm feature representations of the set of training arms; a second determination module 640 configured for determining a target feature representation of the biological target ligand molecule pair based on the updated atomic feature representation; and a model training module 650 configured for training a ligand generative model using the target feature representation of the biological target ligand molecule pair.

In some embodiments, the third decomposition module 620 is further configured for: dividing a plurality of atoms in the training ligand molecule into a plurality of atom clusters based on a binding between each atom in the training ligand molecule and a local binding pocket of the training target, the plurality of atom clusters including a set of arm atom clusters and a skeleton atom cluster; and determining the set of training arms based on the set of arm clusters.

In some embodiments, the apparatus 600 further comprises a feature determining module configured for constructing a first atomic neighbor graph based on a distance from a plurality of atoms in the training target to an atom in the first training arm, wherein an atomic connection in the first atomic neighbor graph indicates that a distance between two atoms is less than a first preset distance; and determining a first graph feature representation of the first atomic neighbor graph as the first arm feature representation of the first training arm.

In some embodiments, the feature updating module 640 is further configured for: for a second training arm of the set of training arms: determining a set of atoms corresponding to the second training arm in the training ligand molecule; and concatenating the second arm feature representation of the second training arm to an atomic feature representation of the set of atoms as an atomic feature representation of the set of atomic updates.

In some embodiments, the feature updating module 640 is further configured for: constructing a second atomic neighbor graph based on a distance between a plurality of atoms in the biological target ligand molecule pair, wherein an atomic connection in the second atomic neighbor graph indicates that a distance between two atoms is less than a second preset distance; and determining a second graph feature representation of the second atomic neighbor graph as a target feature representation of the biological target ligand molecule pair.

In some embodiments, the ligand generation model is a diffusion model.

The modules and/or units included in apparatus 500 and/or apparatus 600 may be implemented in various ways, including software, hardware, firmware, or any combination thereof. In some embodiments, one or more modules and/or units may be implemented using software and/or firmware, such as machine-executable instructions stored on a storage medium. In addition to or as an alternative to machine-executable instructions, some or all of the modules and/or units in apparatus 500 and/or apparatus 600 may be implemented, at least in part, by one or more hardware logic components. By way of example and not limitation, exemplary types of hardware logic components that may be used include field programmable gate arrays (FPGAs), application specific integrated circuits (ASICs), application specific standards (ASSPs), system-on-a-chip (SOCs), complex programmable logic devices (CPLDs), and the like.

FIG. 7 illustrates a block diagram of an electronic device 700 in which one or more embodiments of the present disclosure may be implemented. It should be understood that the electronic device 700 illustrated in FIG. 7 is merely exemplary and should not constitute any limitation on the functionality and scope of the embodiments described herein. The electronic device 700 shown in FIG. 7 may be configured to implement the training device 120 and/or the generation device 140 in FIG. 1.

As shown in FIG. 7, the electronic device 700 is in the form of a general-purpose computing device. Components of the electronic device 700 may include, but are not limited to, one or more processors or processing units 710, a memory 720, a storage device 730, one or more communication units 740, one or more input devices 750, and one or more output devices 760. The processing unit 710 may be an actual or virtual processor and capable of performing various processes according to programs stored in the memory 720. In multiprocessor system, multiple processing units execute computer-executable instructions in parallel to improve parallel processing capabilities of electronic device 700.

Electronic device 700 typically includes a plurality of computer storage media. Such media may be any available media accessible to the electronic device 700, including, but not limited to, volatile and non-volatile media, removable and non-removable media. The memory 720 may be volatile memory (e.g., registers, caches, random access memory (RAM)), non-volatile memory (e.g., read-only memory (ROM), electrically erasable programmable read-only memory (EEPROM), flash memory), or some combination thereof. Storage device 730 may be a removable or non-removable medium and may include a machine-readable medium, such as a flash drive, magnetic disk, or any other medium, which may be capable of storing information and/or data (e.g., training data for training) and may be accessed within electronic device 700.

The electronic device 700 may further include additional removable/non-removable, volatile/non-volatile storage media. Although not shown in FIG. 7, a disk drive for reading or writing from a removable, nonvolatile magnetic disk (e.g., a “floppy disk”) and an optical disk drive for reading or writing from a removable, nonvolatile optical disk may be provided. In these cases, each drive may be connected to a bus (not shown) by one or more data media interface. The memory 720 may include a computer program product 725 having one or more program modules configured to perform various methods or actions of various embodiments of the present disclosure.

The communication unit 740 is configured to communicate with another electronic device through a communication medium. Additionally, the functionality of components of the electronic device 700 may be implemented in a single computing cluster or multiple computing machines capable of communicating over a communication connection. Thus, the electronic device 700 may operate in a networked environment using logical connections with one or more other servers, network personal computers (PCs), or another network Node.

The input device 750 may be one or more input devices, such as a mouse, a keyboard, a trackball, or the like. The output device 760 may be one or more output devices, such as a display, a speaker, a printer, or the like. The electronic device 700 may also communicate with one or more external devices (not shown) through the communication unit 740 as needed, external devices such as storage devices, display devices, etc., communicate with one or more devices that enable a user to interact with the electronic device 700, or communicate with any device (e.g., a network card, a modem, etc.) that enables the electronic device 700 to communicate with one or more other electronic devices. Such communication may be performed via an input/output (I/O) interface (not shown).

According to example embodiments of the present disclosure, there is provided a computer-readable storage medium having computer-executable instructions stored thereon, wherein the computer-executable instructions are executed by a processor to implement the method described above. According to an exemplary embodiment of the present disclosure, a computer program product is further provided, the computer program product being tangibly stored on a non-transient computer readable medium and comprising computer executable instructions, the computer executable instructions being executed by a processor to implement the method described above.

Aspects of the present disclosure are described herein with reference to flowcharts and/or block diagrams of methods, apparatuses, devices, and computer program products implemented in accordance with the present disclosure. It should be understood that each block of the flowchart and/or block diagram, and combinations of blocks in the flowcharts and/or block diagrams, may be implemented by computer readable program instructions.

These computer-readable program instructions may be provided to a processing unit of a general purpose computer, special purpose computer, or other programmable data processing apparatus to produce a machine, such that the instructions, when executed by a processing unit of a computer or other programmable data processing apparatus, produce means to implement the functions/acts specified in the flowchart and/or block diagram. These computer-readable program instructions may also be stored in a computer-readable storage medium that cause the computer, programmable data processing apparatus, and/or other devices to function in a particular manner, such that the computer-readable medium storing instructions includes an article of manufacture including instructions to implement aspects of the functions/acts specified in the flowchart and/or block diagram(s).

The computer-readable program instructions may be loaded onto a computer, other programmable data processing apparatus, or other apparatus, such that a series of operational steps are performed on a computer, other programmable data processing apparatus, or other apparatus to produce a computer-implemented process such that the instructions executed on a computer, other programmable data processing apparatus, or other apparatus implement the functions/acts specified in the flowchart and/or block diagram block or blocks.

The flowchart and block diagrams in the figures show architecture, functionality, and operation of possible implementations of systems, methods, and computer program products according to various implementations of the present disclosure. In this regard, each block in the flowchart or block diagram may represent a module, program segment, or portion of an instruction that includes one or more executable instructions for implementing the specified logical function. In some alternative implementations, the functions noted in the blocks may also occur in a different order than noted in the figures. For example, two consecutive blocks may actually be performed substantially in parallel, which may sometimes be performed in the reverse order, depending on the functionality involved. It is also noted that each block in the block diagrams and/or flowchart, as well as combinations of blocks in the block diagrams and/or flowchart, may be implemented with a dedicated hardware-based system that performs the specified functions or actions, or may be implemented in a combination of dedicated hardware and computer instructions.

Various implementations of the present disclosure have been described above, which are exemplary, not exhaustive, and are not limited to the implementations disclosed. Many modifications and variations will be apparent to those of ordinary skill in the art without departing from the scope and spirit of the various implementations illustrated. The selection of the terms used herein is intended to best explain the principles of the implementations, the practical application, or improvements to the technology in the marketplace, or to enable others of ordinary skill in the art to understand the various embodiments disclosed herein.

I/we claim:

1. A method for generating a ligand molecule, comprising:
determining a set of initial arms by decomposing a reference ligand molecule for a target biological target;
generating a set of candidate ligand molecules based on the set of initial arms;
determining a set of candidate arms corresponding to each of the set of initial arms by decomposing each of the set of candidate ligand molecules;
determining a target candidate arm for each initial arm from the set of candidate arms to determine a set of target candidate arms; and
generating a set of ligand molecules for the target biological target based on the set of target candidate arms.

2. The method of claim 1, wherein generating a set of candidate ligand molecules based on the set of initial arms comprises:
providing the set of feature representations of the set of initial arms to a ligand generative model to generate the set of candidate ligand molecules, wherein the ligand generation model is a trained diffusion model.

3. The method of claim 1, wherein the reference ligand molecule comprises:
a preset ligand molecule for the target biological target; or
a reference ligand molecule generated based on the target biological target according to another ligand molecules generation process.

4. The method of claim 1, wherein determining the set of initial arms comprises:
dividing a plurality of atoms in the reference ligand molecule into a plurality of atom clusters based on a binding between each atom in the reference ligand molecule and a local binding pocket of the target, the plurality of atom clusters comprising a set of arm atom clusters and a skeleton atom cluster; and
determining the set of initial arms based on the set of arm clusters.

5. The method of claim 1, wherein determining a target candidate arm for each initial arm from the set of candidate arms comprises:
determining the target candidate arm for each initial arm from the set of candidate arms based on a property of the set of candidate arms.

6. The method of claim 5, wherein the property comprises at least one of: affinity, drug resistance, and synthesizability.

7. The method of claim 1, wherein the set of candidate ligand molecules comprise a first set of candidate ligand molecules, the set of candidate arms comprise a first set of candidate arms, and generating a set of ligand molecules for the target biological target based on the set of target candidate arms comprises:
iteratively performing the following steps until a preset condition is satisfied:
generating a second set of candidate ligand molecules based on the set of target candidate arms;
determining a second set of candidate arms corresponding to each of the set of initial arms by decomposing each of the second set of candidate ligand molecules;
determining a target candidate arm for each initial arm from the second set of candidate arms to replace the set of target candidate arms;
generating the set of ligand molecules for the target biological target based on the set of target candidate arms after iteration.

8. The method of claim 7, wherein the preset condition comprises:
the step is performed a predetermined number of times; or
performance of the second set of candidate ligand molecules is higher than a threshold.

9. A method for constructing a ligand generative model, comprising:
obtaining a biological target ligand molecule pair, the biological target ligand molecule pair comprising a training biological target and a corresponding training ligand molecule;
determining a set of training arms by decomposing the training ligand molecules;
updating an atomic feature representation of a plurality of atoms corresponding to the set of training arms in the training ligand molecule based on a set of arm feature representations of the set of training arms;
determining a target feature representation of the biological target ligand molecule pair based on the updated atomic feature representation; and
training a ligand generative model using the target feature representation of the biological target ligand molecule pair.

10. The method of claim 9, wherein determining a set of training arms by decomposing the training ligand molecules comprises:
dividing a plurality of atoms in the training ligand molecule into a plurality of atom clusters based on a binding between each atom in the training ligand molecule and a local binding pocket of the training target, the plurality of atom clusters including a set of arm atom clusters and a skeleton atom cluster; and
determining the set of training arms based on the set of arm clusters.

11. The method of claim 9, further comprising constructing a first arm feature representation of a first training arm of the set of training arms by:
constructing a first atomic neighbor graph based on a distance from a plurality of atoms in the training target to an atom in the first training arm, wherein an atomic connection in the first atomic neighbor graph indicates that a distance between two atoms is less than a first preset distance; and
determining a first graph feature representation of the first atomic neighbor graph as the first arm feature representation of the first training arm.

12. The method of claim 9, wherein updating the atomic feature representation of the plurality of atoms corresponding to the set of training arms in the training ligand molecule comprises:
for a second training arm of the set of training arms:
determining a set of atoms corresponding to the second training arm in the training ligand molecule; and
concatenating the second arm feature representation of the second training arm to an atomic feature representation of the set of atoms as an atomic feature representation of the set of atomic updates.

13. The method of claim 9, wherein determining the target feature representation of the biological target ligand molecule pair based on the updated atomic feature representation comprises:
constructing a second atomic neighbor graph based on a distance between a plurality of atoms in the biological target ligand molecule pair, wherein an atomic connection in the second atomic neighbor graph indicates that a distance between two atoms is less than a second preset distance; and
determining a second graph feature representation of the second atomic neighbor graph as a target feature representation of the biological target ligand molecule pair.

14. The method of claim 9, wherein the ligand generation model is a diffusion model.

15. An electronic device comprising:
at least one processing unit; and
at least one memory coupled to the at least one processing unit and storing instructions for execution by the at least one processing unit, the instructions, when executed by the at least one processing unit, causing the electronic device to perform actions comprising:
determining a set of initial arms by decomposing a reference ligand molecule for a target biological target;
generating a set of candidate ligand molecules based on the set of initial arms;
determining a set of candidate arms corresponding to each of the set of initial arms by decomposing each of the set of candidate ligand molecules;
determining a target candidate arm for each initial arm from the set of candidate arms to determine a set of target candidate arms; and
generating a set of ligand molecules for the target biological target based on the set of target candidate arms.

16. An electronic device comprising:
at least one processing unit; and
at least one memory coupled to the at least one processing unit and storing instructions for execution by the at least one processing unit, the instructions, when executed by the at least one processing unit, causing the electronic device to perform the actions comprising:
obtaining a biological target ligand molecule pair, the biological target ligand molecule pair comprising a training biological target and a corresponding training ligand molecule;
determining a set of training arms by decomposing the training ligand molecules;
updating an atomic feature representation of a plurality of atoms corresponding to the set of training arms in the training ligand molecule based on a set of arm feature representations of the set of training arms;
determining a target feature representation of the biological target ligand molecule pair based on the updated atomic feature representation; and
training a ligand generative model using the target feature representation of the biological target ligand molecule pair.

17. A computer-readable storage medium having stored thereon a computer program which, when executed by a processor, implements actions comprising:
determining a set of initial arms by decomposing a reference ligand molecule for a target biological target;
generating a set of candidate ligand molecules based on the set of initial arms;
determining a set of candidate arms corresponding to each of the set of initial arms by decomposing each of the set of candidate ligand molecules;
determining a target candidate arm for each initial arm from the set of candidate arms to determine a set of target candidate arms; and
generating a set of ligand molecules for the target biological target based on the set of target candidate arms.

18. A computer-readable storage medium having stored thereon a computer program which, when executed by a processor, implements actions comprising:
obtaining a biological target ligand molecule pair, the biological target ligand molecule pair comprising a training biological target and a corresponding training ligand molecule;
determining a set of training arms by decomposing the training ligand molecules;
updating an atomic feature representation of a plurality of atoms corresponding to the set of training arms in the training ligand molecule based on a set of arm feature representations of the set of training arms;
determining a target feature representation of the biological target ligand molecule pair based on the updated atomic feature representation; and
training a ligand generative model using the target feature representation of the biological target ligand molecule pair.
